(19) United States
(12) Patent Application Publication
(10) Pub. No.: US 2013/0109636 A1
(43) Pub. Date: May 2, 2013

(54) TRI-AMINO RELEASING FULVATE

(71) Applicant: Steven R. Schutt, Prescott, AZ (US)

(72) Inventor: Steven R. Schutt, Prescott, AZ (US)

(21) Appl. No.: 13/356,986

(22) Filed: Jan. 24, 2012

Related U.S. Application Data
(63) Continuation-in-part of application No. 13/285,151, filed on Oct. 31, 2011.
Application No. 13/285,151 is a continuation-in-part of application No. 13/283,730, filed on Oct. 28, 2011, now abandoned.

(51) Int. Cl. A61K 38/06

(52) U.S. Cl. 514/21.9

(57) ABSTRACT

A free amino acid and/or amino ion-releasing molecule useful for a wide variety of medical and cosmetic applications. The chemical name of the new molecule rginino-glycino-parabenzoate Fulvate, sometimes referred to herein as Li-TAFA. Its chemical structure is graphically depicted according to structure VIII

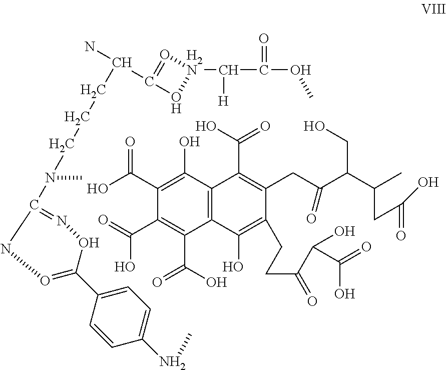

The present invention is a CIP of co-pending application Ser. No. 13/285,151 filed on Oct. 31, 2011 including PCT application PCT/US11/58519; which is a CIP of co-pending application Ser. No. 13/283,730 filed on Oct. 28, 2011; including PCT application PCT/US11/58271 all of which are correspondingly incorporated by reference in their entirety.

### FIELD OF THE INVENTION

The present invention generally relates to amino acids and compounds and complexes made therewith. Many compounds and complexes formed from and between amino acids present significant hydrogen bonding interactions, both internally as well as with the surrounding solvent system.

### BACKGROUND OF THE INVENTION

Amino acids are well known in the art and are taught In most high school curriculums to be an

organic compound containing an amine group (—NH2) which is typically adjacent (or α) to the acid or carboxylic acid (—COOH) moiety. The simplest amino acid is alanine abbreviated as Ala or A and has the graphical representation shown in Structure I.

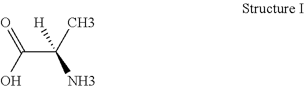

It is common practice in the art when drawing graphical representations of molecules especially organic compounds, to assume a carbon at each vertex, unless another atom is shown, and to assume the presence of the number of hydrogens needed to complete the carbons octet, which is four bonds. Following this guideline, alanine would be drawn as in Structure II.

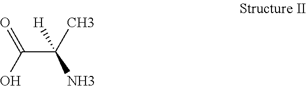

Arginine is an amino acid and is abbreviated ARG or R and is depicted graphically as Structure III. Arginine is produced In the human body through various metabolic pathways including the digestion of proteins. Arginine is also known as 2-amino-S-guanidinovaleric acid yet, according to the International Union of Pure and Applied Chemistry naming system should be named as the amino acid preferably. Arginine is commercially available from numerous sources, including Sigma Aldrich of St. Louis Mo. USA.

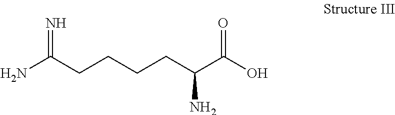 [SMILES: N=C(N)CCCC[C@H](N)C(=O)O]

PABA is an acronym representing Para-Amino Benzoic Acid and is depicted graphically In Structure IV. PABA IS typically a white free-flowing solid available from numerous commercial sources, such as Sigma-Aldrich of St. Louis, Mo., USA. PABA is known to provide several health benefits, is a powerful uv absorber and free-radical scavenger. Owing to the para orientation between the carboxylic acid moiety and the amino group, PABA is a strong hydrogen bonding partner and often found in ionically bonded interactions.

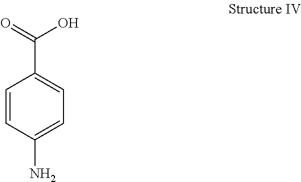

In 1995 applicant filed a patent application describing a compound called arginine aminobenzoate made by combining arginine with PABA. U.S. Pat. Nos. 5,734,080, 6,365,167 and 6,585,988 were issued based on disclosures relating to the 1995 original application. Arginine benzoate was made by combining PABA in isopropanol at 60° C. with agitation, to which was added the slurry made from the addition of arginine (either 1 or d or a mixture thereof) to isoproponal also at 60° C. The resultant mixture was filtered to yield the desired reaction product.

Glycine is the simplest amino acid, is the only one absent a chiral center adjacent the amine and is shown graphically as Structure V.

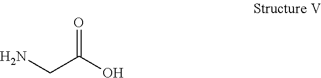

According to IUPAC nomenclature, glycine is also known as amino-acetic acid and is available as a dietary supplement from numerous commercial sources, including Sigma Aldrich of St. Louis, Mo., USA. As a dietary supplement, glycine is reported to provide numerous health benefits including antacid treatment, general antibiotic and gastric and bladder lavage agent.

Carbohydrate derived Fulvic Acid (hereinafter “CHDFA”) has the general molecular structure depicted in FIG. VII with C₂₇H₂₆O₁₈ as the molecular formula, including 18 hydrogen bond acceptors and 10 hydrogen bond donors. CHDFA is produced through the wet oxidation of refined sucrose, using medical grade oxygen and water purified through reverse osmosis.

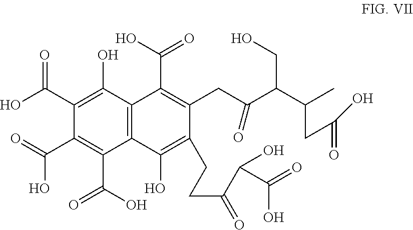 [SMILES: CC(CC(=O)O)C(CO)C(=O)Cc1c(CCC(=O)C(O)C(=O)O)c(O)c2c(C(=O)O)c(C(=O)O)c(C(=O)O)c(O)c2c1C(=O)O]

CDFA has several highly polar and polarizable functional groups, providing ample sites for powerful hydrogen bond and ionic interactions. Perhaps through these bonding sites, CHDFA is known to be a powerful chelating and ionic binding agent. CHDFA has been shown capable of transporting many beneficial amino acids and essential minerals across skin and other membranes. Through these powerful ionic interactions CHDFA forms a stable complex with the amino acids para-Amino benzoic acid, arginine, glycine and alanine forming a complex represented by FIG. VIII

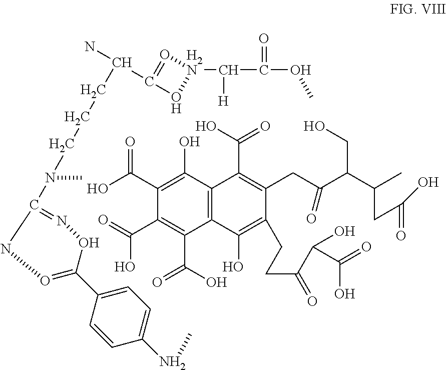

Those of ordinary skill in the art will have recognized the inclusion of various cations, such as Li as counter ions to the described complexes. Without excluding the use of other cations as claimed, the present invention is further directed to the use of Lithium as a counter-ion, thereby providing Li-TAFA, or Lithium-Triamino Fulvate.

Lithium treatment is used to treat mania in bipolar disorder. Initially, lithium is often used in conjunction with antipsychotic drugs as it can take up to a month for lithium to have an effect. Lithium is also used as prophylaxis for depression and mania in bipolar disorder. It is sometimes used for other psychiatric disorders such as cycloid psychosis and major depressive disorder. Lithium possesses a very important antisuicidal effect not shown in other stabilizing medications such as antiseizures drugs. Non-psychiatric applications are limited; however, its use is well established in the prophylaxis of some headaches related to cluster headaches (trigeminal autonomic cephalgias), particularly hypnic headache. An Italian pilot study in humans conducted in 2005-06 suggested that lithium may improve outcomes in the neurodegenerative disease amyotrophic lateral sclerosis. However, a randomized, double-blind, placebo-controlled trial comparing the safety and efficacy of lithium in combination with riluzole for treatment of amyotrophic lateral sclerosis failed to demonstrate any benefit from a combination therapy over riluzole alone.

Lithium is sometimes used as an augmenting agent to increase the benefits of standard drugs used for unipolar depression. Lithium treatment was previously considered to be unsuitable for children; however, more recent studies show its effectiveness for treatment of early-onset bipolar disorder in children as young as eight. The required dosage (15-20 mg per kg of body weight) is slightly less than the toxic level, requiring blood levels of lithium to be monitored closely during treatment. To prescribe the correct dosage, the patient's entire medical history, both physical and psychological, is sometimes taken into consideration. The starting dosage of lithium should be 400-600 mg given at night and increased weekly depending on serum monitoring.

Those who use lithium should receive regular serum level tests and should monitor thyroid and kidney function for abnormalities. As it interferes with the regulation of sodium and water levels in the body, lithium can cause dehydration. Dehydration, which is compounded by heat, can result in increasing lithium levels. The reason why water is lost is because Lithium inhibits the action of antidiuretic hormone (ADH) which enables the kidney to reabsorb water from urine. This causes an inability to concentrate urine leading to consequent loss of body water and thirst.

High doses of haloperidol, fluphenazine, or flupenthixol may be hazardous when used with lithium; irreversible toxic encephalopathy has been reported.

Lithium salts have a narrow therapeutic/toxic ratio and should therefore not be prescribed unless facilities for monitoring plasma concentrations are available. Patients should be carefully selected. Doses are adjusted to achieve plasma concentrations of 0.4 to 1.2 mmol Li⁺/L (lower end of the range for maintenance therapy and elderly patients, higher end for pediatric patients) on samples taken 12 hours after the preceding dose. Overdosage, usually with plasma concentrations over 1.5 mmol Li⁺/L, may be fatal, and toxic effects include tremor, ataxia, dysarthria, nystagmus, renal impairment, confusion, and convulsions. If these potentially hazardous signs occur, treatment should be stopped, plasma lithium concentrations redetermined, and steps taken to reverse lithium toxicity. The most common side effects are an overall dazed feeling and a fine hand tremor. These side effects are generally present during the length of the treatment, but can sometimes disappear in certain patients. Other common side effects, such as nausea and headache, can be generally remedied by a higher intake of water. Lithium unbalances electrolytes; to counteract this, increased water intake is suggested.

Lithium toxicity is compounded by sodium depletion. Concurrent use of diuretics that inhibit the uptake of sodium by the distal tubule (e.g. thiazides) is hazardous and should be avoided. In mild cases, withdrawal of lithium and administration of generous amounts of sodium and fluid will reverse the toxicity. Plasma concentrations in excess of 2.5 mmol Li⁺/L are usually associated with serious toxicity requiring emergency treatment. When toxic concentrations are reached, there may be a delay of 1 or 2 days before maximum toxicity occurs.

In long-term use, therapeutic concentrations of lithium have been thought to cause histological and functional changes in the kidney. The significance of such changes is not clear, but is of sufficient concern to discourage long-term use of lithium unless it is definitely indicated. Doctors may change a bipolar patient's medication from lithium to another mood stabilizing drug, such as valproate (Depakote), if problems with the kidneys arise. An important potential consequence of long-term lithium usage is the development of renal diabetes insipidus (inability to concentrate urine). Patients should therefore be maintained on lithium treatment after 3-5 years only if, on assessment, benefit persists. Conventional and sustained-release tablets are available. Preparations vary widely in bioavailability, and a change in the formulation used requires the same precautions as initiation of treatment. There are few reasons to prefer any one simple salt of lithium; the carbonate has been the more widely used, but the citrate is also available.

Lithium may be used as a treatment of seborrhoeic dermatitis (Lithium gluconate 8% gel). In addition, lithium has been shown to increase production of white blood cells in the bone marrow and might be indicated in patients suffering from leukopenia.

What is needed is a better product for providing free amino ions in the presence of a strong chelating complex including a Lithium counter ion for topical, oral and internal delivery.

### SUMMARY OF THE INVENTION

The present invention provides a lithium salt of free amino ion releasing molecules useful for a variety of medical, cosmetic and cosmeceutical applications. The new lithium complex or molecule according to the present invention is lithio-arginino-glycino-parabenzoate Fulvate, according to IUPAC naming rules. Although the exact molecular orientation is a challenge to determine, one reasonable graphical representation is In Structure VII where the amine in PABA is hydrogen bonded with the acid portion of glycine and the amine portion from arginine is hydrogen bonded with the acid portion of the PABA and lithium is the counter-ion (not shown) In this representation, the nitrogen's are drawn without hydrogen's, to more clearly demonstrate the ionic interactions.

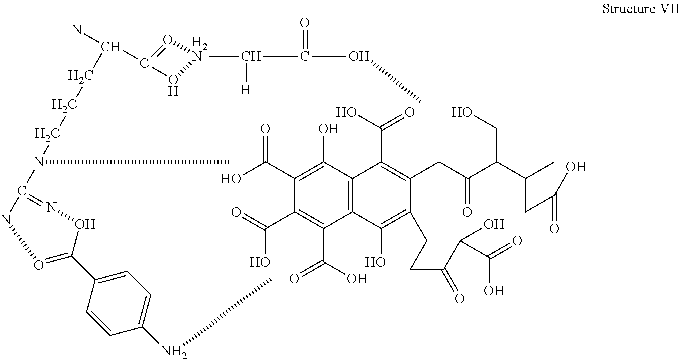

The new lithium and amino acid releasing complex can be formed according to either of two methods. The first method includes the steps of mixing the lithium salt of any or all of the amino acids used, arginine and glycine in water at about room temperature, to provide the lithium glycine-arginate salt intermediate as expected according to traditional acid-base chemical understanding. The intermediate is then combined with PABA and CHDFA to yield the final composition. The second method inverts the order whereby PABA and CHDFA are combined with glycine to form an intermediate Lithio-para-aminobenzo-glycin-fulvate, according to IUPAC naming guidelines. This intermediate is then combined with arginine to provide the desired product, lithio-arginino-glycino-parabenzene-fulvate, according to IUPAC naming rules, but which shall be referred to as “Lithium-tri-amino fulvate” and/or “Li-TAFA” In this disclosure and is graphically represented as Structure VIII. Of course the individual molecules are able to adopt a more energy beneficial orientation, perhaps influenced by temperature or other ionic interactions yet maintaining the essential molecular formula of one PABA, one arginine one glycine and one CHDFA and whereas lithium is the cationin counter ion of choice, as further depicted in Structure VIII.

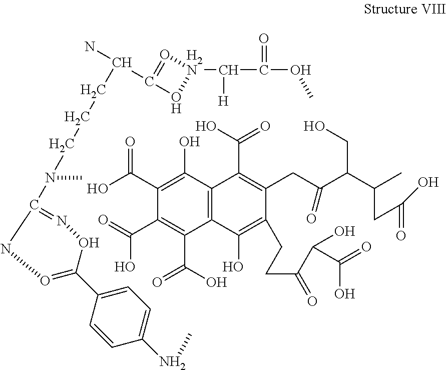

### DETAILED DESCRIPTION

Applicants believe that unstable nucleic acids within mutant cell nuclei cause or at least facilitate the further mutation of those cells. It is also felt that when dysfunctional cells, whose nuclei contain unstable nucleic acids, react with free amino ions or free ionic amino acids In an aqueous solution or an aqueous environment the resulting biosynthesis causes alignment and stabilization of those cells. Applicant asserts it is the process, at least in part, of the free amino ion and/or the free amino acid hydrogen bonding and/or ionically bonding to the unstable nucleic acids in the nuclei within the mutant cells, promotes this alignment and resultant stabilization. One further beneficial result of this re-alignment is the unexpected result of those cells to return to normal function, meaning to function according to similar or analogous cells without the dysfunction. The biosynthesis occurs when a free amino ion and/or a free amino acid interacts ionically with the unstable nucleic acid resulting In stabilization and alignment. Applicant asserts this is why and how amniotic fluid keeps the proteins within fetal cell clusters stable. Specifically, there is present in amniotic fluid an overwhelming amount of free amino and/or free ionic amino acids causing constant biosynthesis and thereby cellular alignment. First process for preparing the arginino glycino-parabenzene-fulvate-lithium salt, or Li-TAFA is through the production of an Intermediate reaction product useful in releasing free amino acid ions, which because of weak internal hydrogen bonding, allows for the release of amino ions. These ions provide numerous and various positive physiological effects In both humans and animals such as stabilizing unstable nucleic acids within dysfunctional cells by process of biosynthesis. This reaction product may also be used to boost and accelerate topical microcirculation and is also useful In the manufacture of other reaction products. These additional reaction products allow for release of a greater quantity of free amino ions to facilitate positive physiological effects.

To obtain Li-TAFA, those skilled In the art will dissolve at ultra high shear and at 80° Centigrade, 227.0 Grams of arginine (either d or L or a mixture thereof) in 1 liter of de-ionized water (USP). The ultra high shear mixing should take at least four minutes. Applicant uses a Ross homogenizer; however a Fischer PowerGen Model 1800D homogenizer would work satisfactorily. The mixing speed should be al least 4,000 rpm. The resulting solution should have pH of about 11.5. Reduce the mixing speed to 300 rpm and add 183.0 Grams of aminoacetic acid. Gradually increase mixing speed to 4000 rpm and mix at this speed for at least four minutes. pH should be about 9.2 but will rise to about 9.3 as solution cools, combine thereto at least a 1 molar equivalent of CHDFA, continue mixing for another 60 seconds and allow solution to stand. This a satisfactory end point for the Li-TAFA complex and is ready for use in various topical, oral, nasal and internal applications.

To desiccate Li-TAFA base solution to powdered form: Place the Li-TAFA base solution in a Pyrex evaporating dish and then into vacuum oven. Set temperature to no more than 95° C. with a vacuum of between 1 and 1.5 negative atmospheres; evaporate until anhydrous. The resultant material will be crystalline. Remove from oven, cool and grind to a fine powder (4 microns or finer). The finished product will be a white powder.

### Experimental Results

Described below are a few examples and associated physical and biological responses to use of the present invention.

Li-TAFA has exhibited vasodilatation qualities and may be used topically or internally to accelerate microcirculation without toxic side effects. This acceleration of microcirculation has many positive benefits. Wound Healing coupled with the restoration of cellular function, Li-TAFA has exhibited the ability to aid and accelerate the healing of wounds, burns (of all degrees) and several types of chronic lesions. Amongst those lesions healed were radiation burns of the 3rd degree with complete suppression of scar formation), squamous cell carcinoma, basal cell carcinoma, psoriasis, venous stasis ulcers, decubitus ulcers (bedsores), deep surgical wounding (with suppression of scar formation) oral and genital herpes kidney issues, itching, and bleeding secondary to skin lesions.

### Hypertension

Encapsulated Li-TAFA has demonstrated the ability to reduce hypertension without toxic side effects.

### Hair Restoration

The application of TAFA, which has been incorporated into a lotion has demonstrated the ability to restore follicular function (hair growth).

### Anti-Inflammatory

Li-TAFA in all forms as shown the ability to reduce inflammation and control the discomfort resulting from inflammation.

### Pain Relief

Li-TAFA is not an anesthetic, yet has demonstrated substantial pain relief, without diminishing topical sensation which is of great importance in treating wounds such as burns and skin lesions.

### Prostate

Li-TAFA has demonstrated the ability to shrink a swollen prostate to normal size.

### Inhalation Product

To prepare a mixture for therapeutic inhalation via small volume nebulizer, use a 1:1 dilution of Li-TAFA to 50% de-ionized water; combine 1 lite of the Li-TAFA water mixture to 5 grams caffeine and 1 gm xylitol.

While there have been shown what are presently considered to be preferred embodiments of the present invention, it will be apparent to those skilled in the art that various changes and modifications can be made herein without departing from the scope and spirit of the invention. For example, persons skilled in the art may be enabled based on the disclosures herein to recognize additional techniques for producing the intermediate reaction products and/or the final reaction product described herein. Applications of the reaction products (in addition to the ones suggested herein) will undoubtedly become apparent to persons skilled in the art. However, the scope of the patent should be determined by the appended claims and their legal equivalence and not by the examples which have been given.

1. A composition consisting essentially of:
an arginine a glycine an alanine a para-amino benzoate and a carbohydrate derived fulvic acid graphically depicted according to structure VIII.

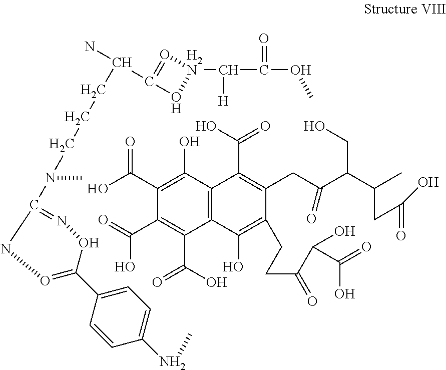

2. The composition according to claim 1 wherein there is between about 0.01 to about 20% molar excess PABA.

3. The composition according to claim 1 wherein there is between about 0.01 to about 80% molar excess CHDFA.

4. The composition according to claim 1 wherein there is between about 0.01 to about 20% molar excess alanine.

5. The composition according to claim 1 wherein there is between about 0.01 to about 20% molar excess arginine.

6. The composition according to claim 1 wherein at least one nitrogen is charged.

7. The composition according to claim 1 wherein at least one oxygen atom is charged.

8. The composition according to claim 1 wherein the compound further includes at least one metal.

9. The composition according to claim 8 wherein the at least one metal is an ion.

10. The composition according to claim 9 wherein the at least one metal is selected from period 4, 5 or 6 of the periodic table.

11. The composition according to claim 10 wherein the at least one metal is Li.

12. The composition according to claim 8 wherein the at least one metal is chelated thereto.
